(19) United States
(12) Patent Application Publication
(10) Pub. No.: US 2024/0197508 A1
(43) Pub. Date: Jun. 20, 2024

(54) DELIVERY MECHANISM FOR REPOSITIONABLE INTRACRANIAL STENT

(71) Applicant: Medical Device Imagineering LLC, Somerset, NJ (US)

(72) Inventors: Jin PARK, Morris Plains, NJ (US); Daniel OLSEN, Califon, NJ (US); Cory ZEGEL, Hoboken, NJ (US)

(21) Appl. No.: 18/537,888

(22) Filed: Dec. 13, 2023

Related U.S. Application Data
(60) Provisional application No. 63/432,851, filed on Dec. 15, 2022.

(51) Int. Cl. A61F 2/966

(52) U.S. Cl. CPC A61F 2/966; A61F 2002/9665

(57) ABSTRACT

A stent delivery system for in-situ deployment of a self-expanding stent is described. The stent delivery system includes a self-expanding stent retention sleeve and a pushwire disposed inside the self-expanding stent retention sleeve. The self-expanding stent retention sleeve includes a proximal end, a distal end, and a central section having a plurality of slots that form a plurality of struts in the central section. The distal end of the pushwire has at least one groove formed therein, which releasably couples with at least one protrusion formed at a proximal end of the self-expanding stent. The distal end of the self-expanding stent retention sleeve moves toward the proximal end of the self-expanding stent retention sleeve when the self-expanding stent retention sleeve is in an unconstrained configuration, thereby releasing the at least one protrusion from the at least one groove to deploy the self-expanding stent.

### CROSS REFERENCE TO RELATED APPLICATIONS

This application claims the priority benefit of U.S. Provisional Application No. 63/432,851, filed Dec. 15, 2022, the entire disclosure of which is hereby incorporated herein in its entirety by reference.

### TECHNICAL FIELD

The present invention is generally related to the field of intravascular therapeutic devices and delivery systems, and more specifically to expandable stents and their corresponding delivery systems. More particularly, this invention is related to very small expandable stents and delivery systems for use in the neurovascular anatomy or similar vascular anatomies for treatment of occluded blood vessels, aneurysms, or other blood vessel disorders.

### BACKGROUND

Blood vessel disorders, specifically those affecting the neurovasculature, including intracranial atherosclerotic disease (ICAD) and aneurysms are a significant point of interest for innovation. Per the Center for Disease Control and Prevention (CDC), in 2020, cerebrovascular diseases were the fifth highest cause of deaths of any cause in the United States.

Stents and similar stenting technology have been successfully used in larger blood vessels in the cardiovascular and peripheral spaces. However, stents and stenting technology that have been used in larger blood vessels, such as the coronary and peripheral anatomy, and have been leveraged for smaller neurovasculature, encounter additional complications due to the smaller diameter of blood vessels in the neurovasculature.

Stenting involves entering a patient's vascular system and deploying a mesh tube to provide structural support to a blood vessel. This may be done to treat blood vessels that have fatty deposits or plaque built up in the blood vessel, otherwise known as atherosclerosis, or to support a vessel that was damaged or whose structural integrity is in question. In the neurovasculature, stenting is most notably used to prevent strokes or treat recurring strokes.

Stents may either be self-expanding or expanded via a mechanical method such as a balloon. As manufactured, stents are loaded onto an appropriate delivery system, either inside of an introducer sheath for a self-expanding stent, or onto a balloon catheter for a non-self-expanding stent. In the conventional procedure, the interventionalist locates the treatment site using a combination of accessories, typically including introducers, guide catheters, guide wires, microcatheters, and fluoroscopic imaging techniques. The interventionalist then inserts the delivery system into a guide catheter or microcatheter appropriately sized for the anatomy being treated. The delivery system with the pre-loaded stent is then advanced to the target location and deployed by either retracting the guide catheter or microcatheter for self-expanding stents or by applying internal pressure to a balloon catheter mechanically, forcing the stent to open, for a non-self-expanding stent.

Stenting in small and tortuous vessels, such as the neurovasculature, introduces additional challenges due to the tortuosity of the anatomy and the reduced size of the vessels. The increased tortuosity of the neurovasculature requires that devices used be more flexible, and the reduced size of the vessels requires that devices and any accessories reduce their size to accommodate the smaller vessels. Due to the small and tortuous vessels of the neurovasculature, devices intended to be used in this part of the anatomy must be specially designed to function appropriately. Special considerations must also be given to the limited use of accessory devices as access of multiple devices simultaneously in the neurovascular may be difficult or impossible.

When stents are being delivered, accuracy of stent placement is critical to ensure that the stent is as effective as possible. In more tortuous anatomies, such as near bifurcations or aneurysms, the accuracy of stent placement is of paramount importance. Should the stent be placed inaccurately, such that it extends beyond the intended vessel wall and into the open vessel, there is significant patient risk for thrombus formation. This risk increases in critical anatomies, such as the neurovasculature, where an improperly placed stent may increase the risk of stroke in the patient. In larger vessels, there are established techniques for improving the accuracy of stent placement using accessory devices, such as additional guidewires or balloon catheters. In the neurovasculature, the reduced vessel diameter and tortuosity either prevents the usage of these techniques or increases their risk and/or difficulty.

One method of providing additional stent placement accuracy is to allow the stent to be recaptured prior to complete deployment. This would provide the interventionalist an opportunity to reposition the stent if it does not deploy accurately in the desired location.

U.S. Pat. No. 7,309,351 discloses a stent which can be recaptured prior to complete deployment. More specifically, U.S. Pat. No. 7,309,351 discloses a self-expanding stent where the ends of the stent are comprised of a strut surrounded by a coil. The delivery system is a coil of variable diameter, where the diameter variation aligns with the stent strut coil diameter. The coiled end of the stent then interacts with the delivery wire to anchor the device in place until the end is unsheathed. However, the stent and stenting technology disclosed in U.S. Pat. No. 7,309,351 introduce two (2) undesirable design limitations. First, by design, the anchor member requires that at least one end of the stent has an attached coil, creating an inconsistency in the stent geometry, which may promote thrombus generation post implantation. Second, in order for the delivery system to be compatible with standard neurovascular accessories (e.g., microcatheter), the stent must have very thin struts to accommodate the additional bulk introduced by the coils on both the stent ends and the mating coil on the delivery system. The thin struts required to make this a viable solution have a severe negative impact on the radial strength of the stent, reducing the overall effectiveness during and after implantation. Although the stent disclosed in U.S. Pat. No. 7,309,351 permits recapture, it is not capable of allowing for stent repositioning while not sacrificing patient outcomes or introducing additional risk.

Therefore, there is a distinct need for a stent and stent delivery system which would improve the accuracy of stent placement in the neurovasculature without the need for accessories or advanced techniques. The desired stent delivery system needs to accurately place a repositionable, low-profile stent which can meet the functional requirements to effectively treat blood vessel disorders without sacrificing patient outcomes or introducing additional risk.

### SUMMARY

At least the above-discussed need is addressed and technical solutions are achieved in the art by various embodiments of the present invention. In some embodiments of the invention, a stent delivery system for in-situ deployment of a self-expanding stent comprises a self-expanding stent retention sleeve, and a pushwire disposed inside the self-expanding stent retention sleeve. The self-expanding stent retention sleeve includes a proximal end, a distal end, and a central section having a plurality of slots forming a plurality of struts. A distal end of the pushwire has at least one groove formed therein, the at least one groove adapted to releasably couple with at least one protrusion formed at a proximal end of the self-expanding stent in a case where the self-expanding stent retention sleeve is in a radially constrained configuration. The distal end of the self-expanding stent retention sleeve is configured to move toward the proximal end of the self-expanding stent retention sleeve in a case where the self-expanding stent retention sleeve transition to an unconstrained configuration from the radially constrained configuration, thereby releasing the at least one protrusion from the at least one groove to deploy the self-expanding stent.

In some embodiments of the invention, the at least one protrusion formed at the proximal end of the self-expanding stent has a keyhole geometry including a first portion and a second portion, the first portion having a larger width than the second portion, the first portion being formed closer to the proximal end of the self-expanding stent retention sleeve than the second portion.

In some embodiments of the invention, the first portion is a circular portion and the second portion is a rectangular portion. In some embodiments of the invention, the at least one groove formed at the distal end of the pushwire has a geometry corresponding to the keyhole geometry of the at least one protrusion formed at the proximal end of the self-expanding stent.

In some embodiments of the invention, the proximal end of the self-expanding stent retention sleeve is affixed to the pushwire such that the proximal end of the self-expanding stent retention sleeve does not move in a longitudinal direction along the pushwire when the self-expanding stent retention sleeve transitions from the radially constrained configuration to the unconstrained configuration. In some embodiments of the invention, the proximal end of the self-expanding stent retention sleeve is welded to the pushwire. In some embodiments of the invention, the proximal end of the self-expanding stent retention sleeve is glued to the pushwire. In some embodiments of the invention, the proximal end of the self-expanding stent retention sleeve is crimped to the pushwire.

In some embodiments of the invention, the stent delivery system further includes a microcatheter. The self-expanding stent retention sleeve is disposed inside the microcatheter. The self-expanding stent retention sleeve is in the radially constrained configuration in a case where the distal end and the central section of the self-expanding stent retention sleeve are covered by the microcatheter. The self-expanding stent retention sleeve transitions to the unconstrained configuration in a case where the microcatheter is retracted toward the proximal end of the self-expanding stent retention sleeve, thereby uncovering the distal end and the central section of the self-expanding stent retention sleeve.

In some embodiments of the invention, the proximal end of the self-expanding stent retention sleeve is formed as a continuous ring, and the distal end of the self-expanding stent retention sleeve is formed as a continuous ring.

In some embodiments of the invention, a length of the self-expanding stent retention sleeve is longer in the case where the self-expanding stent retention sleeve is in the radially constrained configuration than in the case where the self-expanding stent retention sleeve is in the unconstrained configuration.

In some embodiments of the invention, the self-expanding stent retention sleeve prevents the proximal end of the self-expanding stent from being uncoupled from the distal end of the pushwire in the case where the self-expanding stent retention sleeve is in the radially constrained configuration. In some embodiments of the invention, the self-expanding stent retention sleeve permits the proximal end of the self-expanding stent to uncouple from the distal end of the pushwire in the case where the self-expanding stent retention sleeve is in the unconstrained configuration.

In some embodiments of the invention, the self-expanding stent is recapturable so long as the self-expanding stent retention sleeve is in the radially constrained configuration.

In some embodiments of the invention, the stent delivery system further includes at least one radiopaque or fluoroscopic element disposed on at least one of the pushwire or the self-expanding stent retention sleeve.

In some embodiments of the invention, the distal end of the self-expanding stent retention sleeve covers the at least one groove formed in the pushwire and releasably coupled to the at least one protrusion formed at the proximal end of the self-expanding stent in the case where the self-expanding stent retention sleeve is in the radially constrained configuration, to permit the self-expanding stent to be recaptured within the stent delivery system. In some embodiments of the invention, the distal end of the self-expanding stent retention sleeve retracts towards the proximal end of the self-expanding stent retention sleeve, permitting the at least one groove formed in the pushwire to uncouple from the at least one protrusion formed at the proximal end of the self-expanding stent in the case where the self-expanding stent retention sleeve transitions to the unconstrained configuration, thereby fully deploying the self-expanding stent.

In some embodiments of the system, a stent delivery system for in-situ deployment of a self-expanding stent comprises a self-expanding stent retention sleeve and a pushwire disposed inside the self-expanding stent retention sleeve. The self-expanding stent retention sleeve includes a proximal end, a distal end, and a central section having a plurality of slots forming a plurality of struts. The proximal end of the self-expanding stent is held in place via compression of the self-expanding stent retention sleeve against the pushwire. The distal end of the self-expanding stent retention sleeve is configured to move toward the proximal end of the self-expanding stent retention sleeve in a case where the self-expanding stent retention sleeve transitions to an unconstrained configuration from the radially constrained configuration, thereby releasing the compression to deploy the self-expanding stent.

In some embodiments of the system, the distal portion of the pushwire is tapered. In some embodiments of the system, the entirety of the pushwire is tapered.

These and other embodiments of the invention are discussed in detail below.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 depicts a side view of a stent retention sleeve in an undeployed configuration, according to an embodiment of the invention.

FIG. 2 depicts an isometric view of the stent retention sleeve from FIG. 1 in the undeployed configuration, according to an embodiment of the invention.

FIG. 3 depicts a side view of a stent retention sleeve in a deployed configuration, according to an embodiment of the invention.

FIG. 4 depicts an isometric view of the stent retention sleeve from FIG. 3 in the deployed configuration, according to an embodiment of the invention.

FIG. 5 depicts a side view of an alternative stent retention sleeve design in a deployed configuration, according to an embodiment of the invention.

FIG. 6 depicts an isometric view of the alternative stent retention sleeve design from FIG. 5 in the deployed configuration, according to an embodiment of the invention.

FIG. 7 depicts a side view of another alternative stent retention sleeve design in a deployed configuration, according to an embodiment of the invention.

FIG. 8 depicts an isometric view of the another alternative stent retention sleeve design from FIG. 7 in the deployed configuration, according to an embodiment of the invention.

FIG. 9 depicts a side view of another alternative stent retention sleeve design in a deployed configuration, according to an embodiment of the invention.

FIG. 10 depicts an isometric view of the another alternative stent retention sleeve design from FIG. 9 in the deployed configuration, according to an embodiment of the invention.

FIG. 11 depicts a side view of another alternative stent retention sleeve design in a deployed configuration, according to an embodiment of the invention.

FIG. 12 depicts an isometric view of the another alternative stent retention sleeve design from FIG. 11 in the deployed configuration, according to an embodiment of the invention.

FIG. 13 depicts a top view of a proximal end of a stent interfacing with a groove in a pushwire, according to an embodiment of the invention.

FIG. 14 depicts a side view of the proximal end of the stent interfacing with the groove in the pushwire, according to an embodiment of the invention.

FIG. 15 depicts a top view of the proximal end of a stent interfacing with a groove in a pushwire, and radially constrained by the stent retention sleeve in an undeployed configuration, according to an embodiment of the invention.

FIG. 16 depicts an isometric view of the proximal end of the stent interfacing with the groove in the pushwire, and radially constrained by the stent retention sleeve in the undeployed configuration, shown in FIG. 15, according to an embodiment of the invention.

FIG. 17 depicts a side view of a stent delivery system including a proximal end of a stent interfacing with a groove in the pushwire, radially constrained by the stent retention sleeve in an undeployed configuration, and further constrained by a microcatheter to maintain the system in the undeployed configuration, according to an embodiment of the invention.

FIG. 18 depicts a side view of the stent delivery system shown in FIG. 17, with the microcatheter moved proximally away from the proximal end of the stent, but not beyond the point at which the stent retention sleeve has expanded, according to an embodiment of the invention.

FIG. 19 depicts a side view of the proximal end of a stent in an expanded configuration, a stent retention sleeve in an expanded configuration, and associated stent delivery system including a pushwire and a microcatheter, according to an embodiment of the invention.

FIG. 20 depicts a top view of the stent delivery system shown in FIG. 19, according to an embodiment of the invention.

FIG. 21 depicts a top view of a proximal end of a stent secured exclusively through compressive forces between a stent retention sleeve and a pushwire, according to an embodiment of the invention.

FIG. 22 depicts a side view of the proximal end of the stent secured exclusively through compressive forces between the stent retention sleeve and the pushwire, according to an embodiment of the invention.

FIG. 23 depicts a side view of the proximal end of a stent in an expanded configuration, a stent retention sleeve in an expanded configuration, and associated stent delivery system including pushwire and microcatheter, according to another embodiment of the invention.

FIG. 24 depicts a top view of the stent delivery system shown in FIG. 23, according to an embodiment of the invention.

### DETAILED DESCRIPTION

In the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘including at least A’ includes A as well as the possibility of one or more other additional elements besides A. In the same manner, the phrase, ‘including A’ includes A, as well as the possibility of one or more other additional elements besides A. However, the phrase, ‘including only A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The conventional action of stent implantation in a neurovascular vessel typically includes identification of a treatment location by an interventionalist, advancement of a guidewire to the target treatment site, advancement of a microcatheter over the guidewire, removal of the guidewire, then advancement of any devices used in treatment, such as a stent delivery system. For non-self-expanding stents, this occurs using a balloon mounted onto the stent delivery system. For self-expanding stents, this occurs using a wire or equivalent delivery system covered in an introducer sheath, which radially constrains the stent while it is packaged, and until it has entered the microcatheter, at which time the microcatheter radially constrains the stent to prevent premature expansion. Following advancement of the delivery system into the microcatheter, the stent is aligned with the target treatment location and e expanded, either mechanically via a balloon for non-self-expanding stents or by removing the radial constraints upon a self-expanding stent, to allow it to deploy on its own.

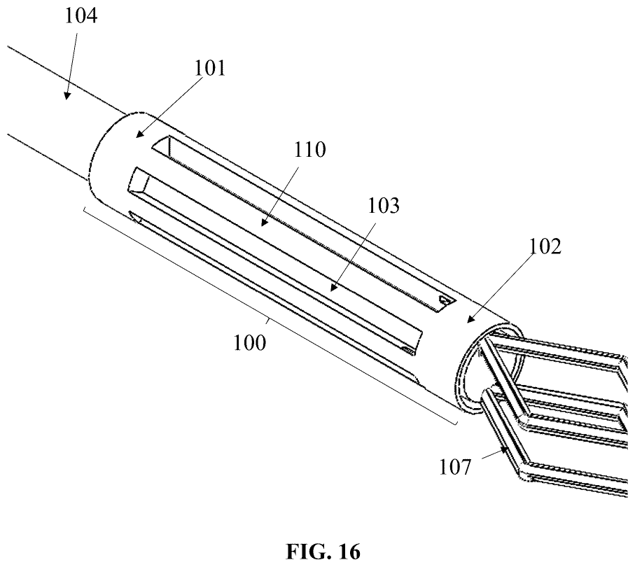

FIG. 16 shows a schematic of a stent delivery system, including a pushwire 104, a stent retention sleeve 100, and a stent 107, according to some embodiments of the invention. The stent delivery system provides a device capable of delivering the stent 107 to a target treatment location in a blood vessel. In FIG. 16, the stent delivery system is in a radially constrained and undeployed configuration, as it would look inside of a microcatheter 108 or introducer sheath.

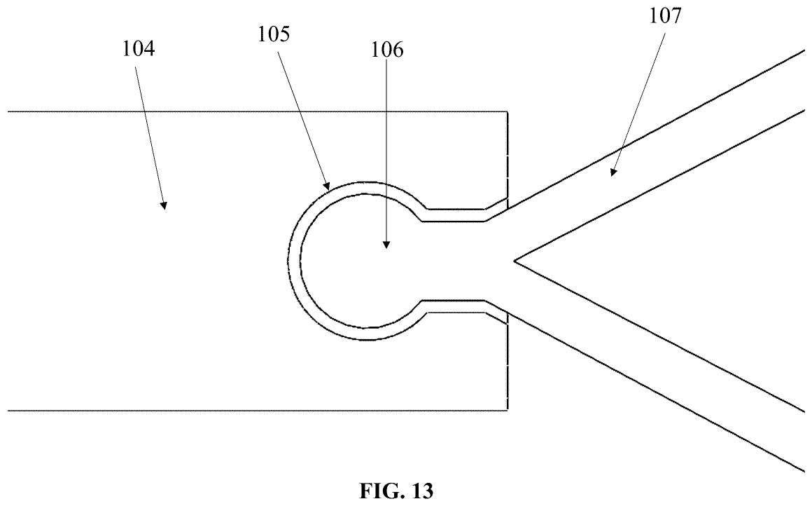

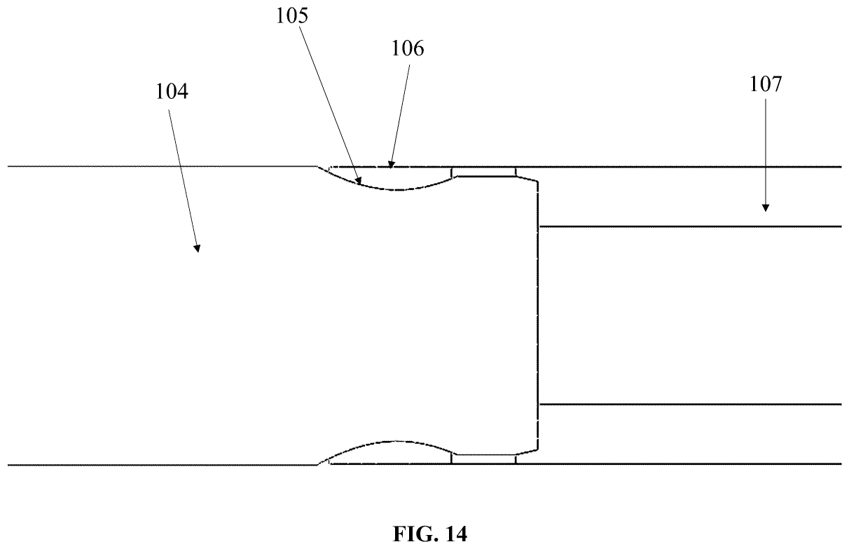

In some embodiments, in the undeployed configuration, the proximal end of the stent 107 is engaged with the pushwire 104 as shown in FIGS. 13 and 14. In some embodiments, the pushwire 104 contains grooves or cutouts 105 adapted to interface with a keyhole geometric portion 106 on a proximal end of the stent 107. In some embodiments of the invention, the keyhole geometric portion 106 is a circular portion attached to a rectangular portion at the proximal end of the stent 107, where a diameter of the circular portion is larger than a width of the rectangular portion. In turn, a shape of the grooves or cutouts 105 corresponds to the outlines of the keyhole geometric portion 106, but all dimensions on the grooves or cutouts 105 are larger than their corresponding geometric portions 106 (i.e. larger diameter circle and larger rectangular strut width). Functionally, this geometric arrangement of the grooves or cutouts 105 and keyhole geometric portions 106 permits the stent 107 to be longitudinally constrained within the delivery system, such that while the keyhole geometric portion 106 is engaged with the grooves or cutouts 105 and the keyhole geometric portion 106 is in a same longitudinal plane as the grooves or cutouts 105, the stent 107 is unable to move longitudinally along the axis corresponding to the centerline of the pushwire 104. In other embodiments of the invention, the keyhole geometric portion 106 and corresponding grooves or cutouts 105 may have a different shape, such as a square, rectangle, wedge, or triangle, with the only constraint being that the width of the rectangular portion of the proximal stent strut is less than the largest width of diameter of the keyhole geometric portion 106. Longitudinally constraining the stent 107 in this manner ensures that the stent 107 can be advanced through the microcatheter 108 when the pushwire 104 is advanced.

In some embodiments of the invention, there is at least one keyhole geometric portion 106 engaged with at least one groove or cutout 105 formed in the pushwire 104.

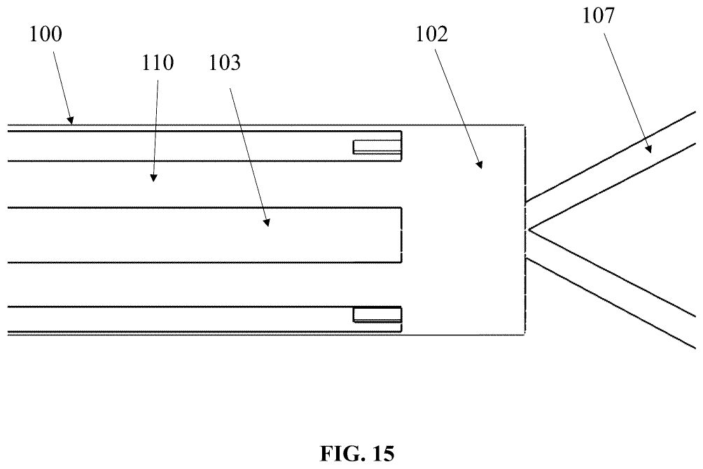

In some embodiments of the invention, the proximal end of the stent 107, comprising the keyhole geometric portion 106, is releasably coupled with the groove 105 in the pushwire 104, and is covered by a stent retention sleeve 100 in a constrained or undeployed configuration, as shown in FIGS. 15 and 16. In some embodiments of the invention, the stent retention sleeve 100 radially constrains the proximal end of the stent 107, preventing the proximal end of the stent 107 from radially expanding while disposed within the sleeve 100. The grooves or cutouts 105 in the pushwire 104 ensure that the proximal end of the stent 107 is fully constrained. This configuration permits the stent 107 to be recaptured during the implantation procedure, so long as the stent retention sleeve 100 is still in the undeployed configuration and the proximal end of the stent 107 and the keyhole geometry 106 is engaged with the grooves or cutouts 105 in the pushwire 104. In some embodiments of the invention, the stent 107 is primarily comprised of a shape memory alloy such as nitinol and is a self-expanding stent.

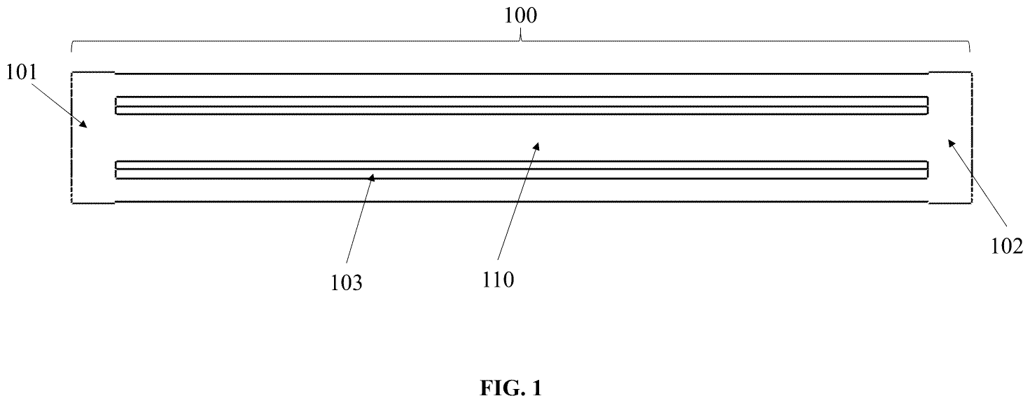

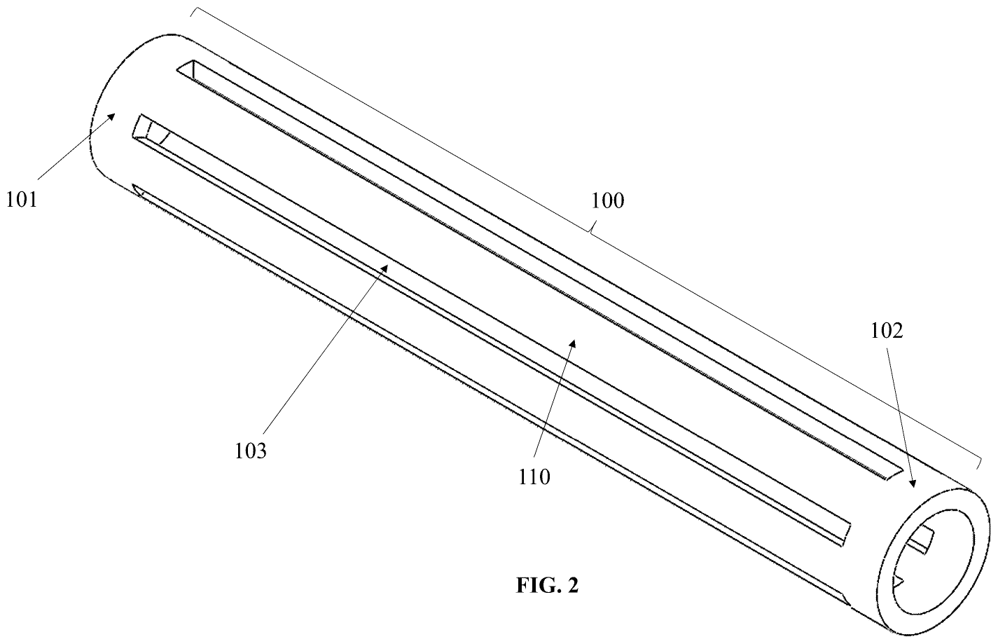

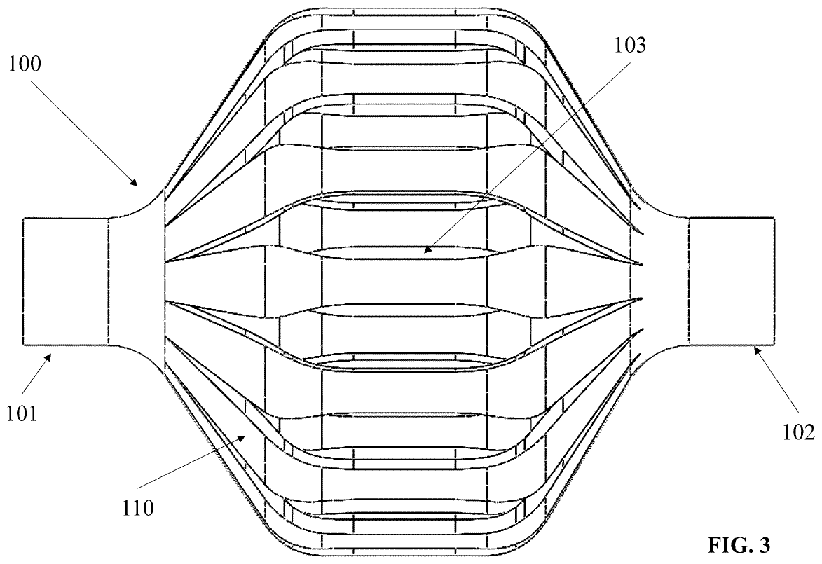

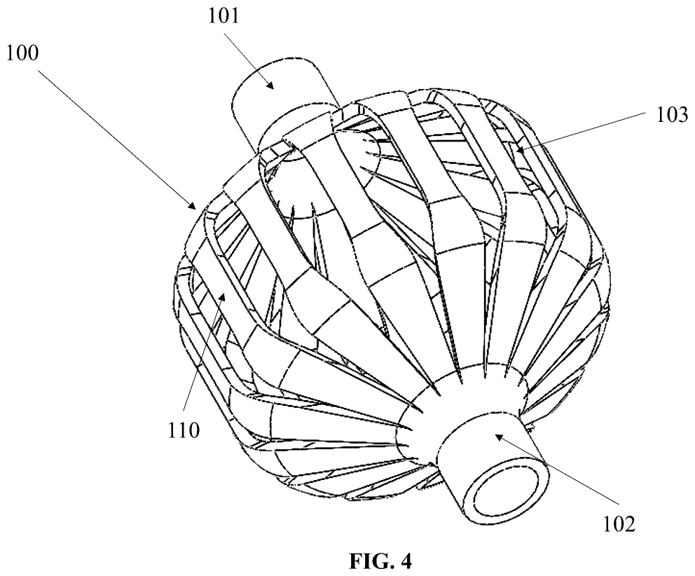

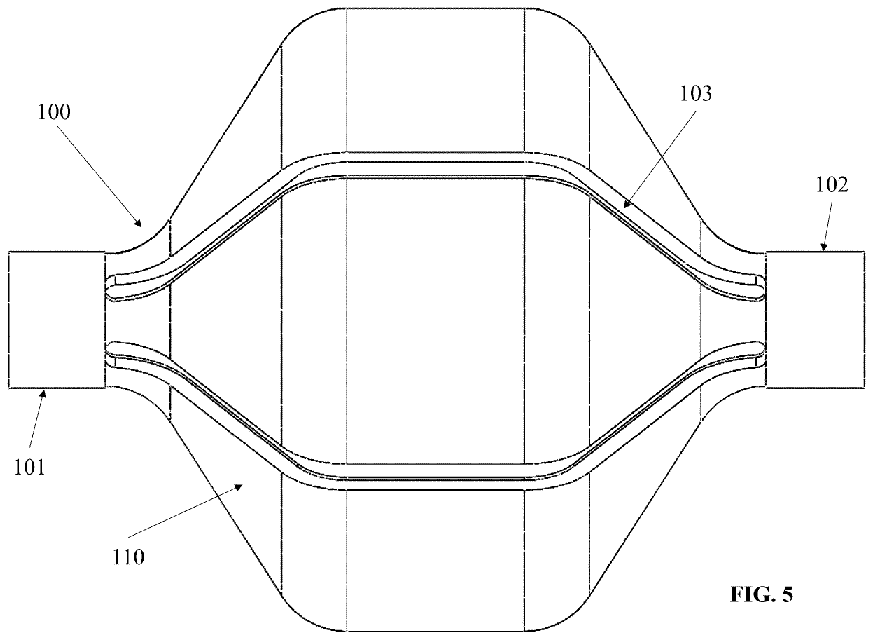

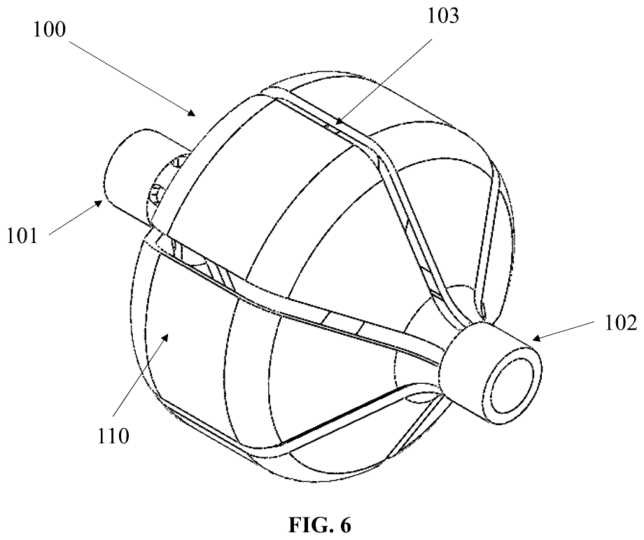

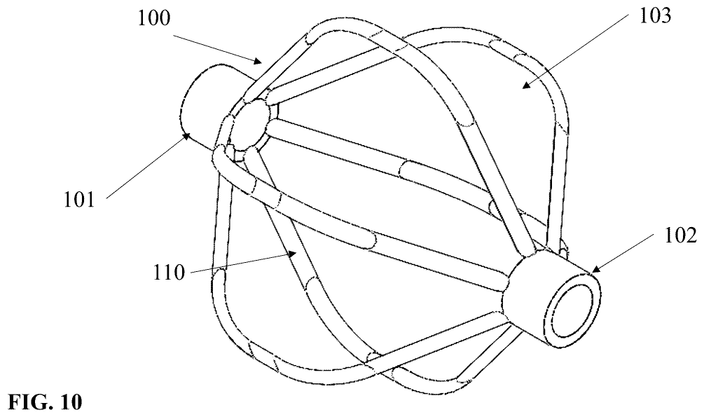

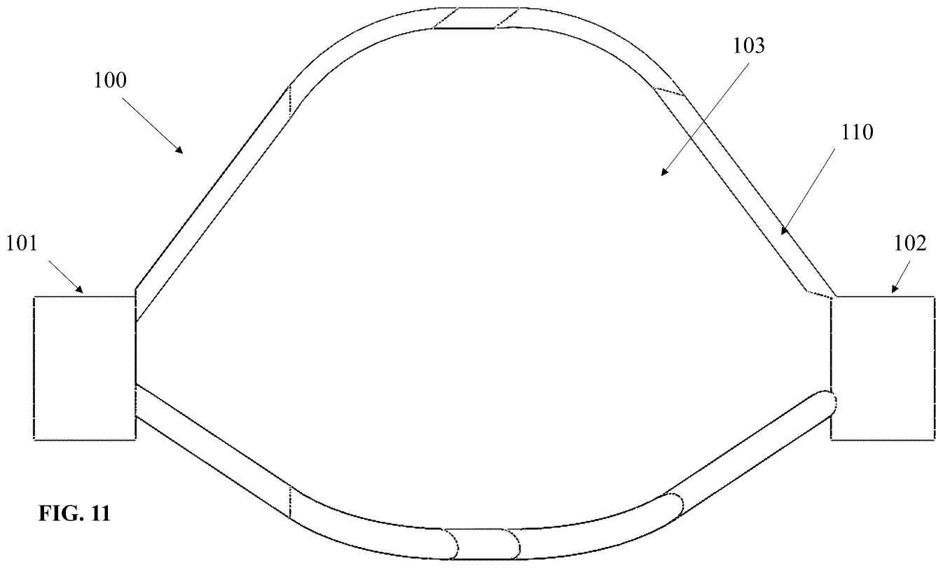

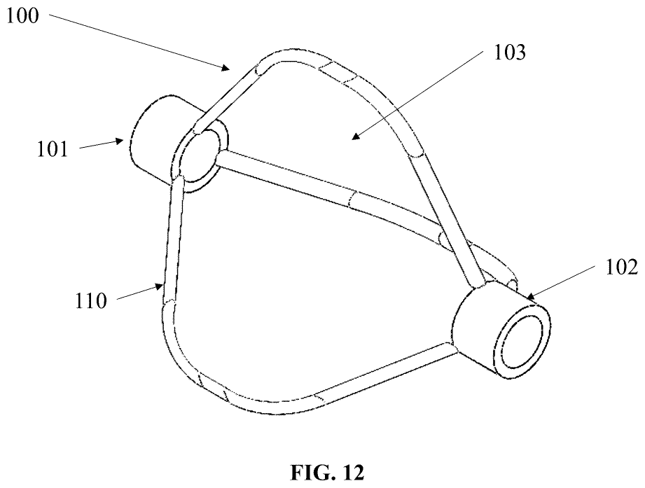

FIG. 1 shows a stent retention sleeve 100 comprising a proximal end 101, a distal end 102, expansion slots 103, and struts 100, according to an embodiment of the invention. The stent retention sleeve 100 may switch between two configurations, either constrained and undeployed as shown in FIGS. 1 and 2, or unconstrained and deployed as shown in FIGS. 3 to 12. According to an embodiment of the invention, the stent retention sleeve 100 is made of a shape memory alloy, such as nitinol. In the radially constrained, or undeployed configuration, the stent retention sleeve 100 is a tube of near-constant diameter with a ring of uncut material on the proximal end 101, a ring of uncut material on the distal end 102, and expansion slots 103 cut along the length of a middle or central section of the stent retention sleeve 100, such that the cuts protrude through the entire surface up to, and not including the proximal end 101 ring and distal end 102 ring. The portions of uncut material along the central section of the stent retention sleeve 100 from struts 110.

In the radially unconstrained, or deployed configuration, the stent retention sleeve 100 maintains the same functional characteristics, including a ring of uncut material on the proximal end 101, a ring of uncut material on the distal end 102, and expansion slots 103 cut along the length of the stent retention sleeve. However, in the deployed configuration, the stent retention sleeve 100 has a variable diameter along the length, with a larger diameter in the center of the stent retention sleeve 100 than on either end 101 or 102. Both the proximal end 101 and distal end 102 are the same diameter, and have a diameter equal to that of the radially constrained or undeployed stent retention sleeve 100. In other words, in the unconstrained or deployed configuration, the proximal end 101 and distal end 102, where no material has been cut, do not expand radially—only the central portion of the stent retention sleeve 100 with the cut expansion slots 103 expands in the unconstrained configuration.

In some embodiments of the invention, the stent retention sleeve 100 has expansion slots 103 which provide struts 110 in the design, permitting the stent retention sleeve 100 to radially expand when unconstrained. In some embodiments of the invention, the expansion slots 103 are rectangular as shown in FIGS. 1 and 2 and FIGS. 5 and 6. In alternative embodiments of the invention, the expansion slots 103 may be tapered or include alternative designs to facilitate the stent retention sleeve 100 to radially expand while retaining its structural integrity. Some examples of various expansion slot 103 configurations are shown in FIGS. 1 through 12. It should be noted, however, that the geometry of the expansion slots 103 is not limited to the examples shown in FIGS. 1 through 12, which are provided for purposes of illustration only. In some embodiments of the invention, the number of expansion slots 103 is dependent upon the desired amount of empty space and the total number of individual struts in the stent retention sleeve 100, and is limited by the diameter of the tube and a minimum size expansion slot 103 that may be cut into the material.

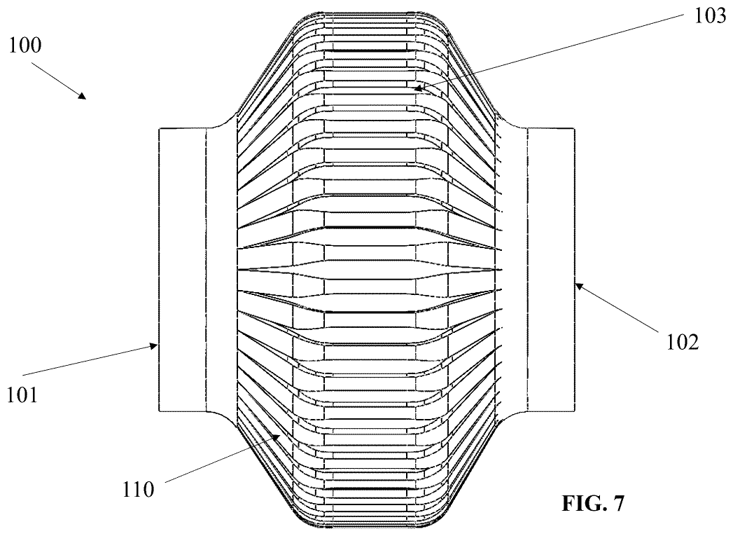

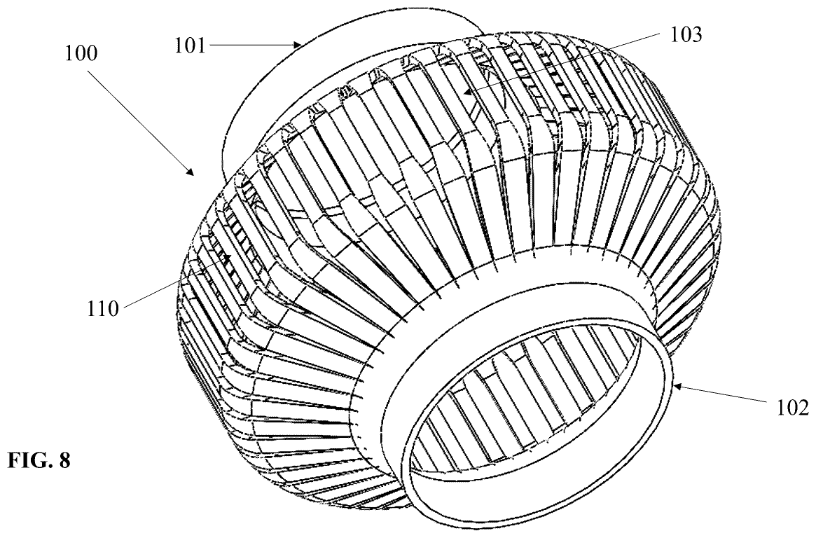

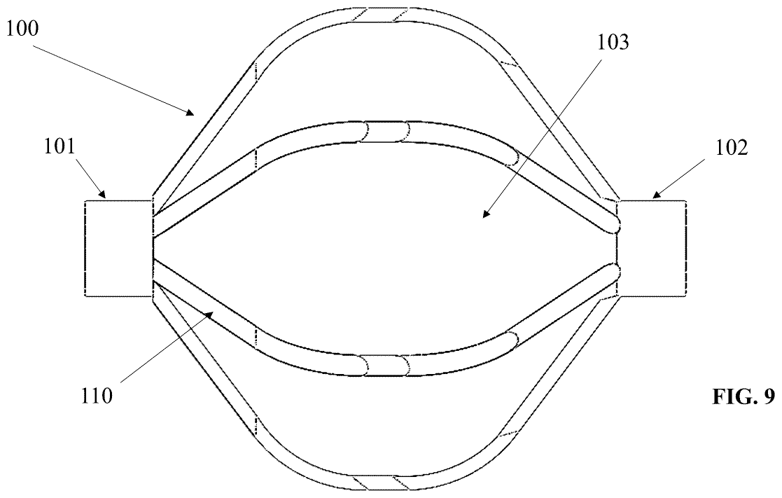

In some embodiments of the invention, the stent retention sleeve 100 is comprised of three sections, a proximal end 101 which is a solid ring, a distal end 12 which is a solid ring, and a middle (central) section that includes the expansion slots 103 forming a plurality of struts 110. The shape of the expansion slots 103 can vary geometrically as shown in FIGS. 3-12. In some embodiments of the invention, the expansion slots 103 have a consistent width along the length of the central section of the stent retention sleeve 100. In some embodiments of the invention, the expansion slots 103 have a variable width along the length of the central section of the stent retention sleeve 100, where the width of the expansion slots 103 increases from the proximal or distal end to the centerline of the stent retention sleeve. In some embodiments of the invention, the width of the expansion slots 103 is very thin, resulting in a large number of struts 110 as shown in FIGS. 7 and 8. In other embodiments of the invention, there are a minimal number of wide expansion slots 103, resulting in a fewer number of struts 110 as shown in FIGS. 9-12. Variations in the shape and width of the expansion slot 103 directly impact the strut shape and width of the stent retention sleeve 100, which impact the radial force and durability of the stent retention sleeve 100. Larger (wider) expansion slots 103 will result in a simpler manufacturing process, but impart lower structural integrity to the stent retention sleeve 100 than smaller (narrower) expansion slots 103.

In some embodiments of the invention, due to the radial expansion of the stent retention sleeve 100, the overall length of the stent retention sleeve 100 is longer in the constrained or undeployed configuration than it is in the unconstrained or deployed configuration. This foreshortening relationship is dependent upon the constructed profile of the stent retention sleeve 100 and the maximum diameter of the stent retention sleeve 100 in the unconstrained or deployed configuration.

In some embodiments of the invention, the proximal end 101 of the stent retention sleeve 100 is welded or similarly attached (e.g., glued, crimped, etc.) to the pushwire 104 to ensure that, when the stent retention sleeve 100 transitions from the constrained (undeployed) configuration to the unconstrained (deployed) configuration, all foreshortening movement of the sleeve 100 occurs from the distal end 102 of the stent retention sleeve 100 and the proximal end 101 of the stent retention sleeve remains fixed with respect to the remainder of the stent delivery system.

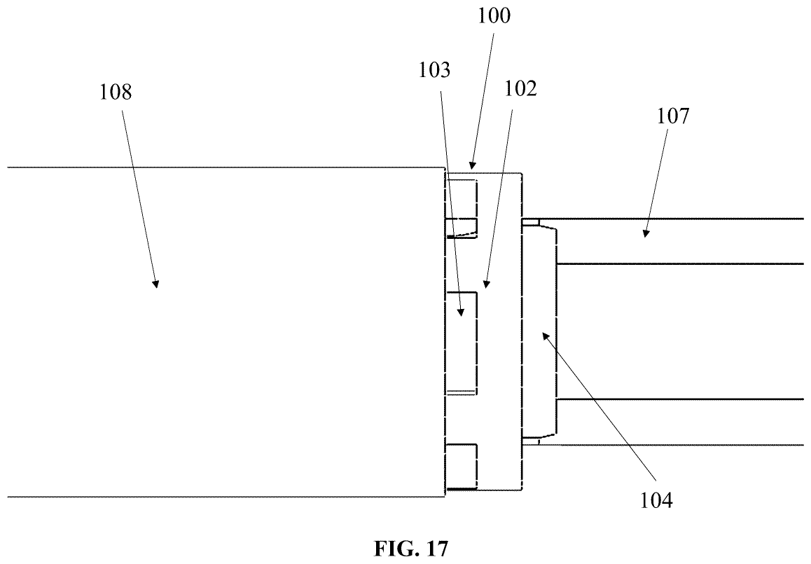

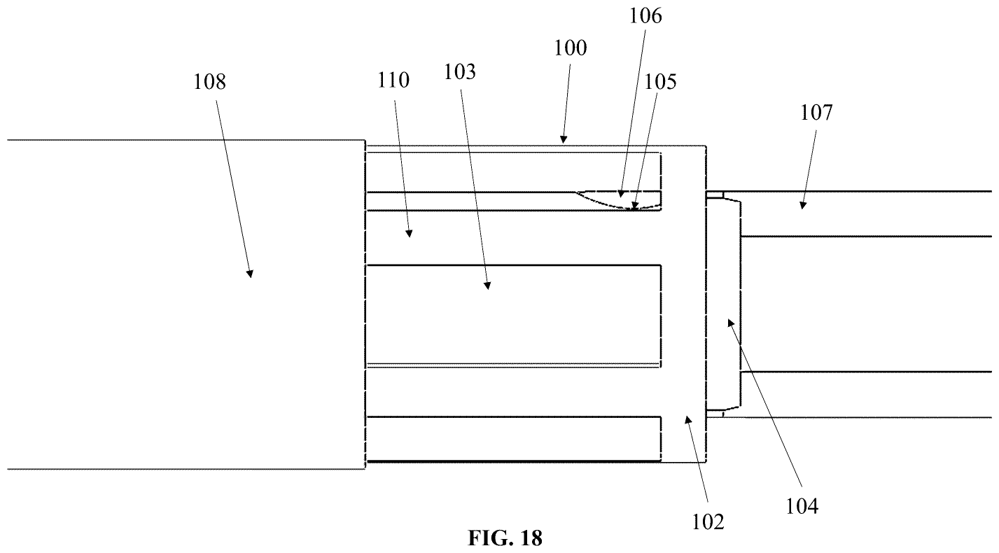

FIGS. 17-19 pictorially depict a deployment process of the stent 107, according to some embodiments of the invention. As shown in FIG. 17, once the stent 107 is at a target location, the microcatheter 108 is partially retracted to expose the stent 107, while the stent 107 and rest of the stent delivery system (including the stent retention sleeve 100) remain stationary. This allows the exposed and now unconstrained sections of the stent 107 to self-expand into the blood vessel. However, as shown in FIG. 17, the stent retention sleeve 100 fully covers the keyhole geometric portion 106 of the proximal end of the stent 107, thereby keeping the proximal end of the stent constrained within the stent delivery system. As shown in FIG. 18, the microcatheter 108 continues to be retracted until just the proximal end 101 of the stent retention sleeve 100 remains covered by the microcatheter 108. At this point, and at any point prior to this, the interventionist may choose to advance the microcatheter 108 back over the sleeve 100 and the stent 107, thereby re-constraining and recapturing the stent 107 fully within the stent delivery system. This permits the interventionist to reposition the stent 107 if the initial location was not satisfactory. This ability of recapture and reposition the statement, even when it is in a mostly-unconstrained configuration with just the proximal end being constrained within the sleeve 100, is especially critical in regions of high tortuosity, or in areas near aneurysms or bifurcations, where the position of the stent is paramount to patient safety.

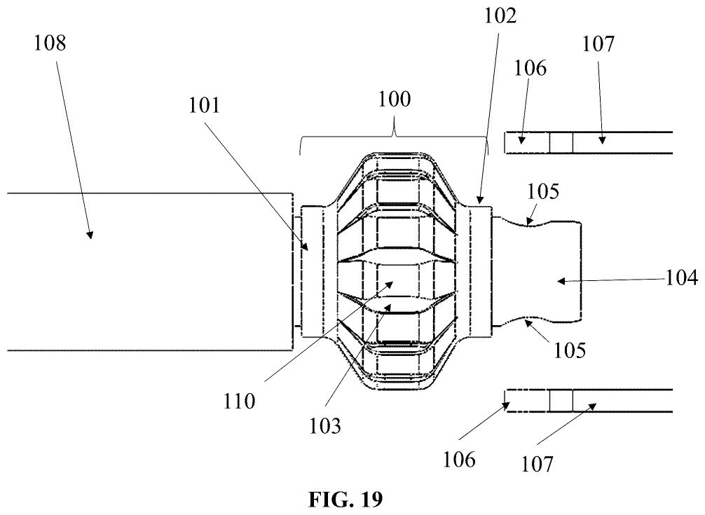

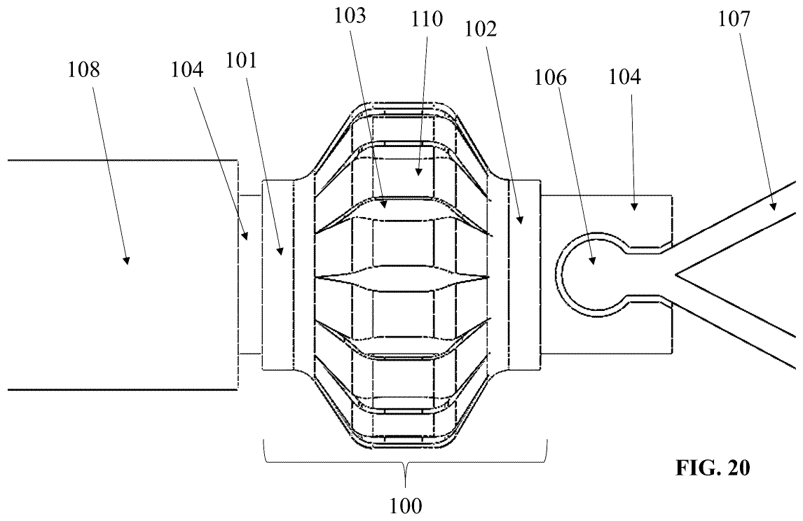

As shown in FIG. 19, if the interventionist is content with the location of the stent 107, they can continue to fully retract the microcatheter 108 until the proximal end 101 of the stent retention sleeve 100 is uncovered, or uncovered to a sufficient degree to allow the central portion of the stent retention sleeve 100, with the expansion slots 103, to radially expand. Once the central portion of the stent retention sleeve 100 is unconstrained, the diameter at the center of the sleeve will increase, thereby foreshortening the stent retention sleeve 100 by drawing the movable distal end 102 closer to the fixed proximal end 101. This foreshortening of the stent retention sleeve 100 causes the distal end 102 of the stent retention sleeve 100 to slide proximally over the keyhole geometric portion 106 until the sleeve retention sleeve 100 no longer radially constrains the keyhole geometric portion 106 on the proximal end of the stent 107. Since there is no radial constraint, the keyhole geometric portion 106 separates from the grooves or cutouts 105, and the proximal end of the stent 107 self-expands into the vessel wall. In this fully expanded configuration, the stent 107 can no longer be recaptured or redeployed. FIGS. 19 and 20 show views of the fully deployed stent 107, in the unconstrained configuration, from two orthogonal directions. As is apparent from FIG. 19, when the solid ring portion 102, formed on the distal end of the stent retention sleeve 100, retracts and moves towards the proximal end 101, the keyhole geometric portions 106 “pop out” of the cutouts 105 formed on the pushwire 104.

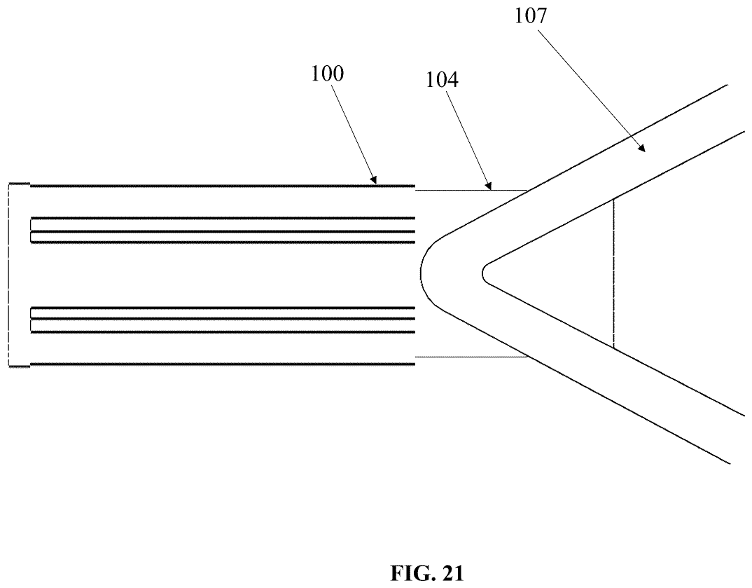

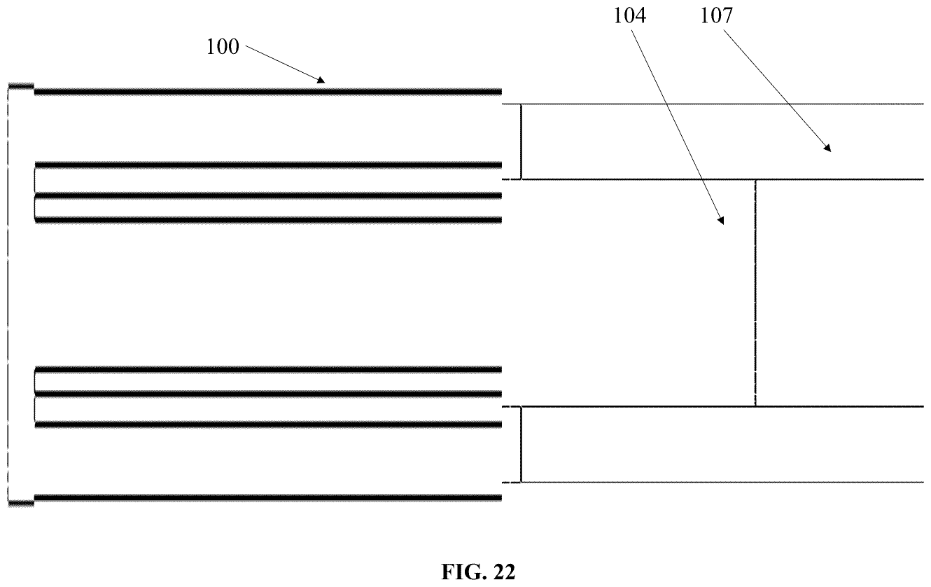

In some embodiments, in the undeployed configuration, the proximal end of the stent 107 is secured exclusively through compressive forces between a stent retention sleeve 100 and a pushwire 104 as shown in FIGS. 21 and 22. FIG. 21 depicts a top view of a proximal end of a stent secured exclusively through compressive forces between a stent retention sleeve and a pushwire, according to an embodiment of the invention. FIG. 22 depicts a side view of the proximal end of the stent secured exclusively through compressive forces between the stent retention sleeve and the pushwire, as shown in FIG. 21, according to an embodiment of the invention.

In some embodiments of the invention, the distal end of the pushwire 104 is tapered to facilitate assembly of the proximal end of the stent 107, the pushwire 104, and the stent retention sleeve 100. In some embodiments, the proximal end of the stent 107 is compressed between the stent retention sleeve 100 and the pushwire 104, which longitudinally constrains the stent 107 within the delivery system, such that while the stent 107 is compressed between the stent retention sleeve 100 and the pushwire 104, the stent 107 is unable to move longitudinally along the axis corresponding to the centerline of the pushwire 104. Longitudinally constraining the stent 107 in this manner ensures that the stent 107 can be advanced through the microcatheter 108 when the pushwire 104 is advanced.

In some embodiments of the invention, the proximal end of the stent 107 is secured exclusively through compressive forces between the stent retention sleeve 100 and the pushwire 104, in a constrained or undeployed configuration. In some embodiments of the invention, the stent retention sleeve 100 radially constrains the proximal end of the stent 107, preventing the proximal end of the stent 107 from radially expanding while disposed within the sleeve 100. The compressive forces ensure that the proximal end of the stent 107 is fully constrained. This configuration permits the stent 107 to be recaptured during the implantation procedure, so long as the stent retention sleeve 100 is still in the undeployed configuration and the proximal end of the stent 107 and is constrained within the sleeve 100. In some embodiments of the invention, the stent 107 is primarily comprised of a shape memory alloy such as nitinol and is a self-expanding stent.

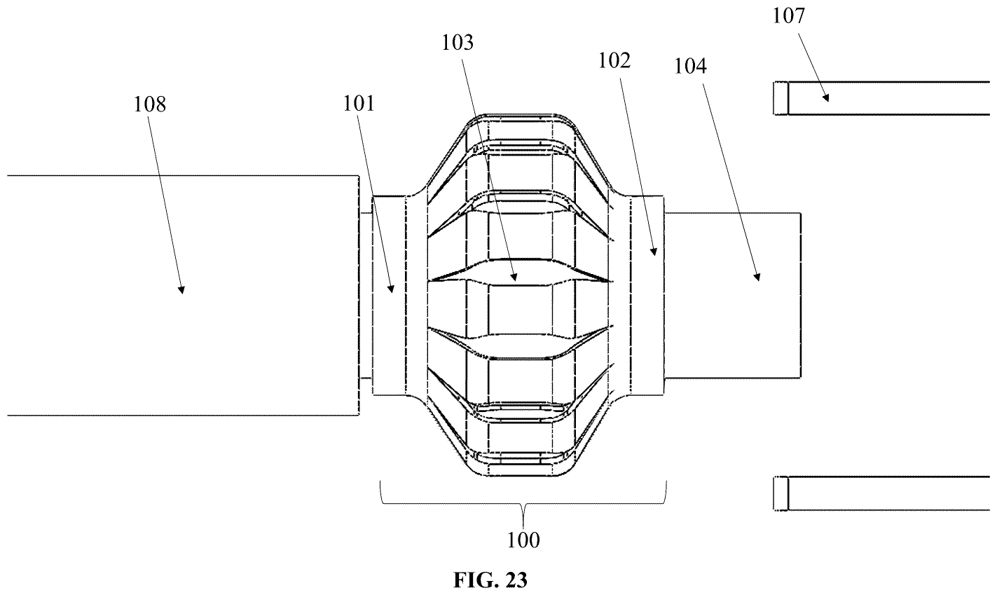

FIGS. 22 and 23 also pictorially depict a deployment process of the stent 107, according to some embodiments of the invention. Similar to FIG. 17, FIG. 22 shows the stent delivery system in a state where the stent 107 is at a target location, and the microcatheter 108 has been partially retracted to expose the stent 107, while the stent 107 and rest of the stent delivery system (including the stent retention sleeve 100) remain stationary. This allows the exposed and now unconstrained sections of the stent 107 to self-expand into the blood vessel. However, as shown in FIG. 22, the stent retention sleeve 100 and the pushwire 104 continue to apply compressive forces to the proximal end of the stent 107, thereby keeping the proximal end of the stent 107 constrained within the stent delivery system. During deployment, the microcatheter 108 continues to be retracted until just the proximal end 101 of the stent retention sleeve 100 remains covered by the microcatheter 108. At this point, and at any point prior to this, the interventionist may choose to advance the microcatheter 108 back over the sleeve 100 and the stent 107, thereby fully re-constraining and recapturing the stent 107 within the stent delivery system. This permits the interventionist to reposition the stent 107 if the initial location was not satisfactory. This ability to recapture and reposition the statement, even when it is in a mostly-unconstrained configuration with just the proximal end being constrained within the sleeve 100, is especially critical in regions of high tortuosity, or in areas near aneurysms or bifurcations, where the position of the stent is paramount to patient safety.

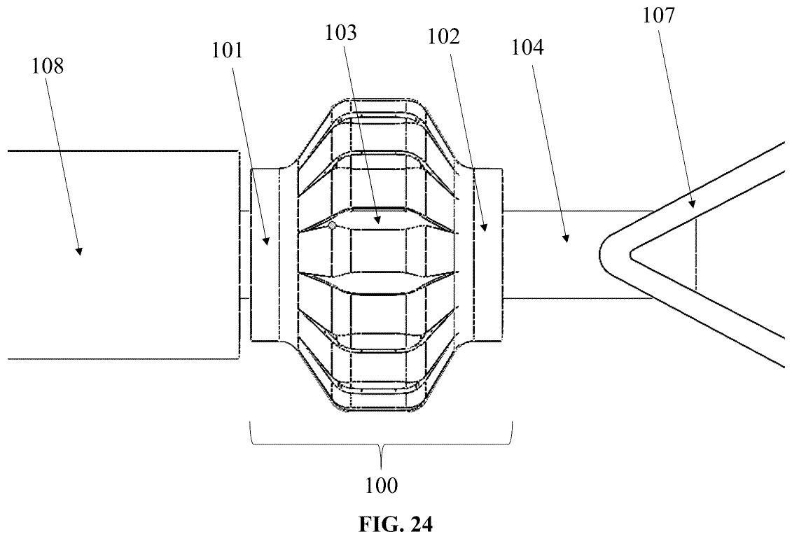

Similar to FIG. 19, and as shown in FIG. 23, if the interventionist is content with the location of the stent 107, they can continue to fully retract the microcatheter 108 until the proximal end 101 of the stent retention sleeve 100 is uncovered, or uncovered to a sufficient degree to allow the central portion of the stent retention sleeve 100, with the expansion slots 103, to radially expand. Once the central portion of the stent retention sleeve 100 is unconstrained, the diameter at the center of the sleeve 100 will increase, thereby foreshortening the stent retention sleeve 100 by drawing the movable distal end 102 closer to the fixed proximal end 101. This foreshortening of the stent retention sleeve 100 causes the distal end 102 of the stent retention sleeve 100 to release the stent 107 since the sleeve 100 no longer radially constrains the proximal end of the stent 107. Since there is no radial constraint, the proximal end of the stent 107 self-expands into the vessel wall. In this fully expanded configuration, the stent 107 can no longer be recaptured or redeployed. FIGS. 23 and 24 show views of the fully deployed stent 107, in the unconstrained configuration, from two orthogonal directions.

In some embodiments of the invention, the stent retention sleeve 100 includes radiopaque materials that provide more precise in-situ location of the stent 107, prior to full deployment. In other embodiments of the invention, the pushwire 104 includes radiopaque materials, such as marker bands, to facilitate visibility for the interventionist. In some embodiments of the invention, the keyhole geometric portion 106 includes a radiopaque element, such as a radiopaque material or marker band.

It should be understood that the invention is not limited to the embodiments discussed above, which are provided for purposes of illustration only. Subsets or combinations of various embodiments described above provide further embodiments of the invention.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. A stent delivery system for in-situ deployment of a self-expanding stent, comprising:
a self-expanding stent retention sleeve; and
a pushwire disposed inside the self-expanding stent retention sleeve;
wherein the self-expanding stent retention sleeve includes:
a proximal end;
a distal end; and
a central section having a plurality of slots forming a plurality of struts,
wherein a distal end of the pushwire has at least one groove formed therein, the at least one groove adapted to releasably couple with at least one protrusion formed at a proximal end of the self-expanding stent in a case where the self-expanding stent retention sleeve is in a radially constrained configuration, and
wherein the distal end of the self-expanding stent retention sleeve is configured to move toward the proximal end of the self-expanding stent retention sleeve in a case where the self-expanding stent retention sleeve transition to an unconstrained configuration from the radially constrained configuration, thereby releasing the at least one protrusion from the at least one groove to deploy the self-expanding stent.

2. The stent delivery system according to claim 1, wherein the at least one protrusion formed at the proximal end of the self-expanding stent has a keyhole geometry including a first portion and a second portion, the first portion having a larger width than the second portion, the first portion being formed closer to the proximal end of the self-expanding stent retention sleeve than the second portion.

3. The stent delivery system according to claim 2,
wherein the first portion is a circular portion and the second portion is a rectangular portion.

4. The stent delivery system according to claim 2,
wherein the at least one groove formed at the distal end of the pushwire has a geometry corresponding to the keyhole geometry of the at least one protrusion formed at the proximal end of the self-expanding stent.

5. The stent delivery system according to claim 1, wherein the proximal end of the self-expanding stent retention sleeve is affixed to the pushwire such that the proximal end of the self-expanding stent retention sleeve does not move in a longitudinal direction along the pushwire when the self-expanding stent retention sleeve transitions from the radially constrained configuration to the unconstrained configuration.

6. The stent delivery system according to claim 5, wherein the proximal end of the self-expanding stent retention sleeve is welded to the pushwire.

7. The stent delivery system according to claim 5, wherein the proximal end of the self-expanding stent retention sleeve is glued to the pushwire.

8. The stent delivery system according to claim 5, wherein the proximal end of the self-expanding stent retention sleeve is crimped to the pushwire.

9. The stent delivery system according to claim 1, further including a microcatheter,
wherein the self-expanding stent retention sleeve is disposed inside the microcatheter,
wherein the self-expanding stent retention sleeve is in the radially constrained configuration in a case where the distal end and the central section of the self-expanding stent retention sleeve are covered by the microcatheter, and
wherein the self-expanding stent retention sleeve transitions to the unconstrained configuration in a case where the microcatheter is retracted toward the proximal end of the self-expanding stent retention sleeve, thereby uncovering the distal end and the central section of the self-expanding stent retention sleeve.

10. The stent delivery system according to claim 1,
wherein the proximal end of the self-expanding stent retention sleeve is formed as a continuous ring, and
wherein the distal end of the self-expanding stent retention sleeve is formed as a continuous ring.

11. The stent delivery system according to claim 1, wherein a length of the self-expanding stent retention sleeve is longer in the case where the self-expanding stent retention sleeve is in the radially constrained configuration than in the case where the self-expanding stent retention sleeve is in the unconstrained configuration.

12. The stent delivery system according to claim 1,
wherein the self-expanding stent retention sleeve prevents the proximal end of the self-expanding stent from being uncoupled from the distal end of the pushwire in the case where the self-expanding stent retention sleeve is in the radially constrained configuration, and
wherein the self-expanding stent retention sleeve permits the proximal end of the self-expanding stent to uncouple from the distal end of the pushwire in the case where the self-expanding stent retention sleeve is in the unconstrained configuration.

13. The stent delivery system according to claim 1, wherein the self-expanding stent is recapturable so long as the self-expanding stent retention sleeve is in the radially constrained configuration.

14. The stent delivery system according to claim 1, further including at least one radiopaque or fluoroscopic element disposed on at least one of the pushwire or the self-expanding stent retention sleeve.

15. The stent delivery system according to claim 1,
wherein the distal end of the self-expanding stent retention sleeve covers the at least one groove formed in the pushwire and releasably coupled to the at least one protrusion formed at the proximal end of the self-expanding stent in the case where the self-expanding stent retention sleeve is in the radially constrained configuration, to permit the self-expanding stent to be recaptured within the stent delivery system, and
wherein the distal end of the self-expanding stent retention sleeve retracts towards the proximal end of the self-expanding stent retention sleeve, permitting the at least one groove formed in the pushwire to uncouple from the at least one protrusion formed at the proximal end of the self-expanding stent in the case where the self-expanding stent retention sleeve transitions to the unconstrained configuration, thereby fully deploying the self-expanding stent.

16. A stent delivery system for in-situ deployment of a self-expanding stent, comprising:
a self-expanding stent retention sleeve; and
a pushwire disposed inside the self-expanding stent retention sleeve;
wherein the self-expanding stent retention sleeve includes:
a proximal end;
a distal end; and
a central section having a plurality of slots forming a plurality of struts,
wherein the proximal end of the self-expanding stent is held in place via compression of the self-expanding stent retention sleeve against the pushwire, and
wherein the distal end of the self-expanding stent retention sleeve is configured to move toward the proximal end of the self-expanding stent retention sleeve in a case where the self-expanding stent retention sleeve transitions to an unconstrained configuration from the radially constrained configuration, thereby releasing the compression to deploy the self-expanding stent.

17. The stent delivery system according to claim 16, wherein the distal portion of the pushwire is tapered.

18. The stent delivery system according to claim 16, wherein the entirety of the pushwire is tapered.
